(19) United States
(12) Patent Application Publication
(10) Pub. No.: US 2024/0257970 A1
(43) Pub. Date: Aug. 1, 2024

(54) SYSTEMS AND METHODS FOR DEEP LEARNING BASED ECG-SIGNAL CLASSIFICATIONS FOR CANINES

(71) Applicant: Mars, Incorporated, McLean, VA (US)

(72) Inventors: André DOURSON, La Wantzenau (FR); Mark PARKINSON, Long Valley, NJ (US); Oliver Roman STIEL, Maidenhead (GB)

(21) Appl. No.: 18/426,581

(22) Filed: Jan. 30, 2024

Related U.S. Application Data
(60) Provisional application No. 63/482,355, filed on Jan. 31, 2023.

(51) Int. Cl. G16H 50/20; G16H 30/20

(52) U.S. Cl. CPC G16H 50/20; G16H 30/20

(57) ABSTRACT

A computer-implemented method for classifying electrocardiogram signals of a canine is disclosed. The method comprises receiving electrocardiogram data of a canine, the electrocardiogram data including at least one electrocardiogram signal, segmenting the electrocardiogram data into one or more data subsets, pre-processing the one or more data subsets, the pre-processing including excluding the one or more data subsets that include a poor electrocardiogram signal, augmenting the one or more data subsets, determining, using a trained machine-learning model, one or more signal classifications for the one or more data subsets, aggregating the one or more signal classifications to determine a result classification, and outputting the result classification to an electronic storage device and/or a display.

### CROSS-REFERENCE TO RELATED APPLICATION(S)

This application claims the benefit of priority to U.S. Provisional Application No. 63/482,355, filed on Jan. 31, 2023, the entirety of which is incorporated herein by reference.

### TECHNICAL FIELD

Various embodiments of this disclosure relate generally to machine-learning based techniques for predicting a classification of electrocardiogram signals of a canine. In some embodiments, the disclosure relates to systems and methods for training a machine-learning based model to predict a classification of electrocardiogram signals of a canine.

### BACKGROUND

Determining a classification of a canine's electrocardiogram (“ECG”) data, such as ECG signals, is an incredibly time consuming process. For example, conventional techniques may include a veterinarian analyzing the canine's ECG signals to determine whether to classify the ECG signals as normal or abnormal. Such techniques are incredibly inefficient, as determining the outcome of the ECG signals is dependent upon the veterinarian's availability. Moreover, conventional techniques fail to distinguish whether the canine's ECG signals are normal or abnormal for the canine's particular breed, age, gender, and the like.

This disclosure is directed to addressing the above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for classifying electrocardiogram data of a canine.

In one aspect, an exemplary embodiment of a method for classifying electrocardiogram signals of a canine is disclosed. The method may include receiving, by one or more processors, electrocardiogram data of a canine, the electrocardiogram data including at least one electrocardiogram signal. The method may further include segmenting, by the one or more processors, the electrocardiogram data into one or more data subsets. The method may further include pre-processing, by the one or more processors, the one or more data subsets, the pre-processing including excluding the one or more data subsets that include a poor electrocardiogram signal. The method may further include augmenting, by the one or more processors, the one or more data subsets. The method may further include determining, by the one or more processors and using a trained machine-learning model, one or more signal classifications for the one or more data subsets. The method may further include aggregating, by the one or more processors, the one or more signal classifications to determine a result classification. The method may further include outputting, by the one or more processors, the result classification to an electronic storage device and/or a display.

In a further aspect, an exemplary embodiment of a computer system for classifying electrocardiogram signals of a canine is disclosed, the computer system comprising at least one memory storing instructions, and at least one processor configured to execute the instructions to perform operations. The operations may include receiving electrocardiogram data of a canine, the electrocardiogram data including at least one electrocardiogram signal. The operations may further include segmenting the electrocardiogram data into one or more data subsets. The operations may further include pre-processing the one or more data subsets, the pre-processing including excluding the one or more data subsets that include a poor electrocardiogram signal. The operations may further include augmenting the one or more data subsets. The operations may further include determining, using a trained machine-learning model, one or more signal classifications for the one or more data subsets. The operations may further include aggregating the one or more signal classifications to determine a result classification. The operations may further include outputting the result classification to an electronic storage device and/or a display.

In a further aspect, a non-transitory computer-readable medium may contain instructions that, when executed by a processor, cause the processor to perform operations for classifying electrocardiogram signals of a canine is disclosed. The operations may include receiving electrocardiogram data of a canine, the electrocardiogram data including at least one electrocardiogram signal. The operations may further include segmenting the electrocardiogram data into one or more data subsets. The operations may further include pre-processing the one or more data subsets, the pre-processing including excluding the one or more data subsets that include a poor electrocardiogram signal. The operations may further include augmenting the one or more data subsets. The operations may further include determining, using a trained machine-learning model, one or more signal classifications for the one or more data subsets. The operations may further include aggregating the one or more signal classifications to determine a result classification. The operations may further include outputting the result classification to an electronic storage device and/or a display.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary flowchart of an exemplary method of training a machine-learning model to predict a classification of electrocardiogram signals of a canine, according to one or more embodiments.

FIG. 2 depicts an exemplary flowchart of an exemplary method of using a machine-learning model to classify electrocardiogram signals of a canine, according to one or more embodiments.

FIG. 3 depicts a flowchart of an exemplary method for predicting a classification of electrocardiogram signals of a canine, according to one or more embodiments.

FIG. 4 depicts an exemplary environment that may be utilized with techniques presented herein, according to one or more embodiments.

FIG. 5 depicts an example of a computing device that may execute the techniques described herein, according to one or more embodiments.

### DETAILED DESCRIPTION OF EMBODIMENTS

According to certain aspects of the disclosure, methods and systems are disclosed for predicting a classification of electrocardiogram (“ECG”) data, such as ECG signals, of a canine. Conventional techniques may not be suitable because conventional techniques may rely on a veterinarian manually examining the ECG data to determine a classification. Such techniques may be inefficient, as they may be dependent upon the availability of a qualified veterinarian. Additionally, conventional techniques may not take the canine's breed, age, or gender into account when analyzing the ECG data. Accordingly, improvements in technology relating to predicting a classification of a canine's ECG data are needed.

ECG data, such as ECG signals, are used for many different purposes in the medical treatment of a canine. For example, the canine's ECG data may be collected and analyzed before the canine may have a surgical procedure. However, conventional methods may require that a veterinarian manually review the collected ECG data to determine that the ECG data is normal, in order for the canine to have the surgical procedure. Such conventional methods are incredibly inefficient and time-consuming, as the conventional methods may depend on a qualified veterinarian's availability. Additionally, conventional methods may also be susceptible to human error.

A need exists for an integrated hardware, software, and machine-learning model solution for classifying a canine's ECG data. Such a solution provides automated support to a cardiologist. Additionally, such a solution may also allow for the automatic classification of the canine's ECG data, without the need for a veterinarian to review the ECG data, while also taking the canine's breed, age, or gender into account.

As will be discussed in more detail below, in various embodiments, systems and methods are described for using a machine-learning model to predict a classification of ECG data of a canine. By training a machine-learning model, e.g., via supervised or semi-supervised learning, to learn associations between a canine's ECG data, at least one signal classification, and/or pet metadata, the trained machine-learning model may be usable to predict a classification of a canine's ECG data. The systems and methods may be able to receive electrocardiogram data of a canine, the electrocardiogram data including at least one electrocardiogram signal. The systems and methods may then be able to segment the electrocardiogram data into one or more data subsets. The systems and methods may then be able to pre-process the one or more data subsets, the pre-processing including excluding the one or more data subsets that include a poor electrocardiogram signal. The systems and methods may then be able to augment the one or more data subsets. The systems and methods may then be able to, upon augmenting the one or more data subsets, determine one or more signal classifications for the one or more data subsets using a trained machine learning model. The systems and methods may then be able to aggregate the one or more signal classifications to determine a result classification. The systems and methods may then be able to output the result classification to an electronic storage device and/or a display.

Presented in this description are various aspects of machine learning techniques that may be adapted to predict a classification of a canine's ECG data. As will be discussed in more detail below, machine learning techniques adapted to predict a classification of a canine's ECG data, may include one or more aspects according to this disclosure, e.g., a particular selection of training data, a particular training process for the machine-learning model, operation of a particular device suitable for use with the trained machine-learning model, operation of the machine-learning model in conjunction with particular data, modification of such particular data by the machine-learning model, etc., and/or other aspects that may be apparent to one of ordinary skill in the art based on this disclosure.

The techniques disclosed in this application are applicable not only to canines, but also to humans and other types of animals, such as domesticated animals like canines, felines, equines, and the like.

### Training a Machine-Learning Model to Classify a Canine ECG Signal

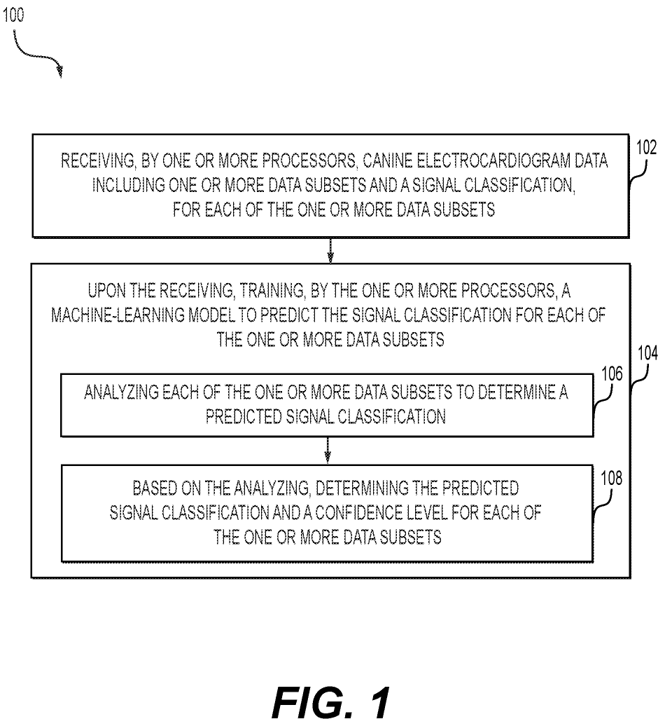

FIG. 1 illustrates an exemplary process for training a machine-learning model to classify electrocardiogram (“ECG”) signals of a canine, according to one or more embodiments. Notably, method 100 may be performed by one or more processors of a server that is in communication with one or more user devices and other external system(s) via a network. However, it should be noted that method 100 may be performed by any one or more of the server, one or more user devices, or other external systems.

The method may include receiving, by one or more processors, canine electrocardiogram data including one or more data subsets and a signal classification for each of the one or more data subsets (Step 102). The receiving may include receiving the one or more data subsets and/or the corresponding signal classifications from one or more user devices. For example, the one or more user devices may collect ECG data of the canine, where the ECG data may include one or more ECG signals. In some embodiments, the receiving may include receiving at least one Digital Imaging and Communications in Medicine (DICOM) file that includes the ECG data. In some embodiments, the DICOM file may be generated by applying one or more sensors to a canine. The one or more sensors may collect ECG data from the canine, and at least one processor may combine the data collected by the one or more sensors into at least one DICOM file. In some embodiments, the one or more data subsets may have been previously augmented. For example, one or more techniques and/or transformations may be applied to the one or more data subsets to optimize, cleanse, and/or standardize the one or more data subsets. The details regarding the techniques and/or transformations are described in the next section.

Additionally, each data subset may be a segmented piece of ECG data. The ECG data may have been segmented into subsets based on one or more factors, such as a signal length, a signal type, and/or a signal interval. The signal length may correspond to a particular time length of the signal. In some embodiments, the signal length may be expressed in milliseconds, seconds, minutes, hours, and the like. For example, the data subset may include an ECG signal length of eight seconds. In another example, the data subset may include an ECG signal length equal to and/or less than ten seconds. In another example, the data subset may include an ECG signal length equal to and/or less than five seconds. In yet another example, the data subset may include an ECG signal length between five seconds and ten seconds. It should be noted that the ECG signal length may be any length that is found to be suitable for a specific application. The signal type may correspond to whether a signal may be an outlier signal or a normal signal. For example, a signal outlier may correspond to one or more signals that exceed a particular threshold, indicating that the one or more signals may be an outlier. In some embodiments, the data subset may include a segment of the ECG data that corresponds to an outlier signal. The signal interval may correspond to one or more ECG waves of the ECG data. For example, the data subset may include a portion of the ECG data that corresponds to an ECG wave.

Each signal classification may indicate a normal classification, an abnormal classification, or a non-diagnostic classification. The normal classification may indicate that the data subset includes normal readings. For example, the data subset readings may be normal if the readings fall within a particular range. The abnormal classification may indicate that the data subset includes abnormal readings. For example, the readings may be abnormal if the readings fall outside of a particular range. The non-diagnostic classification may indicate that there is a possible error, where the data subset does not include normal readings or abnormal readings. In some embodiments, other signal classifications may include an unsuitable classification, a supraventricular classification, a ventricular classification, a bradyarrhythmia classification, and/or a conduction disorder classification. The unsuitable classification may indicate that the signal in the data subset is of poor quality. The supraventricular classification may indicate that signal in data subset shows that the canine's heart's upper chambers are beating too fast. The ventricular classification may indicate that the signal in the data subset shows that the canine's heart's lower chambers are beating too fast. The bradyarrhythmia classification may indicate that the signal in the data subset shows that the canine's heart is beating slower than expected. The conduction disorder classification may indicate an issue with the signal in the data subset, where there may be an issue with the canine's heart's electrical impulses.

The method may also include receiving canine metadata corresponding to the electrocardiogram data. For example, the canine metadata may include at least one of a breed, an age, a gender, a weight, and the like. The canine metadata may be received from a user device and/or from an electronic storage device. For example, one or more displays of a user device may prompt a user to input the canine metadata. Additionally, for example, upon receiving the canine metadata from a user device, an electronic storage device may store the canine metadata (e.g., in a database). In some embodiments, the canine metadata may be received from an electronic storage device that may store metadata corresponding to a canine. For example, the canine metadata may be received from one or more electronic canine medical records of a third party system (e.g., a veterinary system).

Upon receiving the canine electrocardiogram data, the method may include training a machine-learning model to predict the signal classification for each of the one or more data subsets (Step 104). Such training is further described in detail below with respect to Steps 106 and 108. The machine-learning model contemplated in the current disclosure is not limited to a particular model, and may be any model configured to solve the classification problem presented in the current disclosure.

The training may include analyzing each of the one or more data subsets to determine a predicted signal classification (Step 106). For example, the machine-learning model may analyze each data subset and the corresponding signal classification received in the canine electrocardiogram data to determine a predicted signal classification. The predicted signal classification may have the same signal classifications as described above. In some embodiments, the training may also include the machine-learning model analyzing each data subset, the corresponding signal classification, and the canine metadata to determine a predicted signal classification for each of the at least one data subset.

The training may further include, based on the analyzing, determining the predicted signal classification and a confidence level for each of the one or more data subsets (Step 108). For example, the machine-learning model may determine the predicted signal classification and a confidence level based on analyzing each data subset, the corresponding signal classification, and/or the canine metadata. The machine-learning model may determine the confidence level. Additionally, the confidence level may be associated with the machine-learning model's confidence in the predicted signal classification. In some embodiments, the confidence level may be described as a ratio (e.g., 10%).

The method may further include storing the predicted signal classification and/or corresponding confidence level in an electronic storage device. For example, the predicted classification and/or corresponding confidence level may be stored in the electronic storage device for further analysis, machine-learning model training, troubleshooting, and audit.

The method may also include displaying the predicted signal classification and/or the corresponding confidence level determined for each of the data subsets on a display of a user device. In some embodiments, a display of a user device may display at least one of the following predicted signal classifications: a normal classification, an abnormal classification, or a non-diagnostic classification. In some embodiments, a display of a user device may display at least one of the following predicted signal classifications: an unsuitable classification, a supraventricular classification, a ventricular classification, a bradyarrhythmia classification, and/or a conduction disorder classification. In some embodiments, the displaying may include color-coding one or more of the of the data subsets according to the predicted signal classification. For example, if the predicted signal classification for a data subset is an abnormal classification, the data subset may be color-coded red. Additionally, if the predicted signal classification for a data subset is a normal classification, the data subset may be color-coded green. Additionally, if the predicted signal classification for a data subset is a non-diagnostic classification, the data subset may be color-coded blue.

Although FIG. 1 shows example blocks of exemplary method 100, in some implementations, the exemplary method 100 may include additional blocks, fewer blocks, different blocks, or differently arranged blocks than those depicted in FIG. 1. Additionally, or alternatively, two or more of the blocks of the exemplary method 100 may be performed in parallel.

### Using the Trained Machine-Learning Model to Classify a Canine EGG Signal

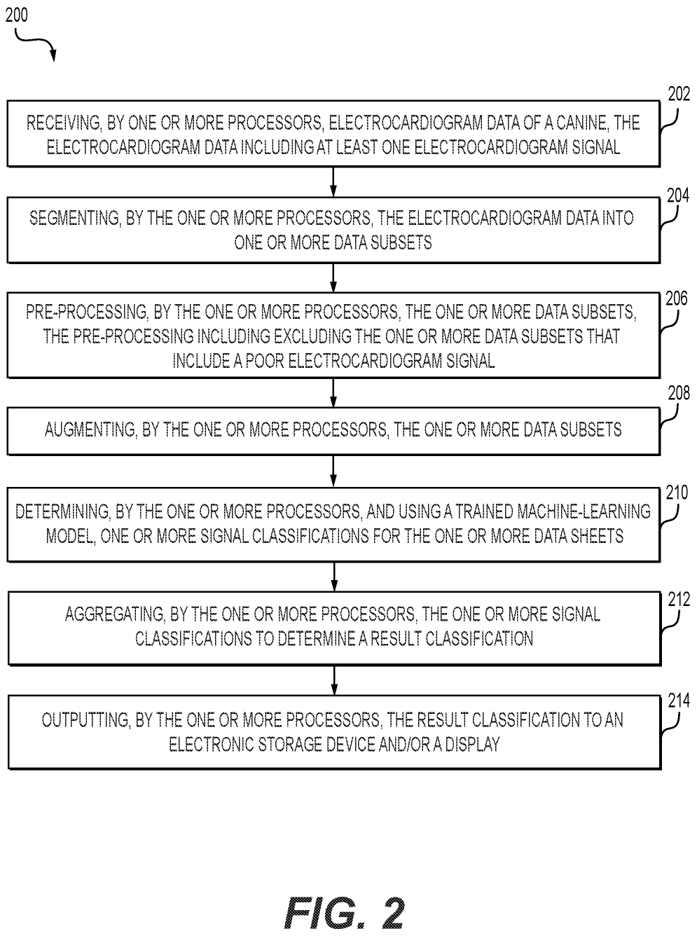

FIG. 2 illustrates an exemplary process for using a trained machine-learning model to classify electrocardiogram (“ECG”) signals of a canine, according to one or more embodiments (e.g., by utilizing a trained machine-learning model such as a machine-learning model trained according to one or more embodiments discussed above). Notably, method 200 may be performed by one or more processors of a server that is in communication with one or more user devices and other external system(s) via a network. However, it should be noted that method 200 may be performed by any one or more of the server, one or more user devices, or other external systems.

The method may include receiving, by one or more processors, electrocardiogram (“ECG”) data of a canine, the electrocardiogram data including at least one electrocardiogram signal (Step 202). The receiving may include receiving the ECG data from one or more user devices. For example, the one or more user devices may collect the ECG data of the canine. In some embodiments, the receiving may include receiving at least one Digital Imaging and Communications in Medicine (DICOM) file that includes the ECG data. In some embodiments, the DICOM file may be generated by applying one or more sensors to a canine. The one or more sensors may collect ECG data, such as at least one ECG signal, from the canine, and at least one processor may combine the data collected by the one or more sensors into at least one DICOM file.

The method may also include, in response to receiving the electrocardiogram data, storing, by the one or more processors, the electrocardiogram data in a data store. For example, the ECG data may be stored in a data store (e.g., a database) for further analysis, machine-learning model training, troubleshooting, and/or audit. The ECG data may also be stored for additional training of machine-learning models.

The method may also include receiving, by the one or more processors, canine metadata corresponding to the electrocardiogram data. For example, the canine metadata may include at least one of a breed, an age, a gender, a weight, and the like. One or more displays of a user device may prompt a user to input the canine metadata. Additionally, for example, upon receiving the canine metadata from a user device, an electronic storage device may store the canine metadata. In some embodiments, the canine metadata may be received from one or more databases that store metadata corresponding to a canine. For example, the canine metadata may be received from one or more electronic canine medical records stored in third party systems (e.g., a veterinary system).

The method may also include segmenting, by the one or more processors, the electrocardiogram data into one or more data subsets (Step 204). The segmenting may include analyzing the ECG data to break the ECG data into one or more data subsets. For example, an electrocardiogram signal may be segmented into one or more sub-segments. The ECG data may be broken into subsets based on one or more factors, which may include a signal length, a signal type, and/or a signal interval. The signal length may correspond to a particular time length of the signal. Additionally, for example, the one or more data subsets may each have the same signal length. In some embodiments, the signal length may be expressed in milliseconds, seconds, minutes, hours, and the like. For example, the one or more data subsets may include an ECG signal length of eight seconds. In another example, a data subset may include an ECG signal length equal to and/or less than ten seconds. In another example, the data subset may include an ECG signal length equal to and/or less than five seconds. In yet another example, the data subset may include an ECG signal length between five seconds and ten seconds. It should be noted that the ECG signal length may be any length that is found to be suitable for a specific application. The signal type may correspond to whether a signal may be an outlier signal or a normal signal. For example, an outlier signal may correspond to one or more signals that exceed a particular threshold, indicating that the one or more signals may be an outlier. For example, the one or more data subsets may include a particular piece of the signal in the ECG data that exceeds a particular threshold. In some embodiments, each of the one or more data subsets may include a segment of the ECG data that corresponds to an outlier signal. The signal interval may correspond to one or more ECG waves of the ECG data. For example, each of the one or more data subsets may include a portion of the ECG data that corresponds to an ECG wave.

The method may also include pre-processing, by the one or more processors, the one or more data subsets, the pre-processing including excluding the one or more data subsets that include a poor electrocardiogram signal (Step 206). The pre-processing may also include determining that at least one data subset has a good ECG signal. In some embodiments, each of the one or more data subsets may be analyzed to determine if the data subset includes a poor electrocardiogram signal. For example, a poor electrocardiogram signal may exceed or fall below a particular threshold. If a data subset includes a poor electrocardiogram signal, such data subset may be excluded from further analysis. The pre-processing the one or more data subsets may further include applying at least one of a baseline wander removal, a signal normalization, a frequency removal, a heart rate computation, and/or an unsuitable signal removal to the one or more data subsets. Similar pre-processing steps may be performed prior to training the machine-learning model as discussed above with respect to FIG. 1.

The method may also include generating, by the one or more processors, a visualization of the one or more data subsets. For example, the visualization may include a millimeter paper look and feel of the one or more data subsets. Such a visualization may be displayed on one or more user interfaces of one or more user devices. Additionally, or alternatively, the visualization may be stored in an electronic storage device.

The method may also include augmenting, by the one or more processors, the one or more data subsets (Step 208). In some embodiments, the augmenting may only be applied to the one or more data subsets that do not include a poor ECG signal. Additionally, for example, one or more techniques and/or transformations may be applied to the one or more data subsets that have the good signal to optimize, cleanse, and/or standardize the one or more data subsets.

The augmenting may include applying, by the one or more processors, at least one transformation to the one or more data subsets. For example, the at least one transformation may include applying at least one of the following to the one or more data subsets that may include part of the ECG signal: randomly shifting a portion of the ECG signal, randomly scaling the ECG signal by a given factor, rolling one random portion of the ECG signal, randomly dropping one consecutive portion of the ECG signal and replacing the porting with a specific number, adding a randomly generated sinusoidal signal to the source signal, adding a randomly generated sinusoidal signal to a random consecutive portion of the source signal, adding a random pulse square signal to the source signal, adding a random pulse square signal to a random consecutive portion of the source signal, adding a random Gaussian noise to the source signal, converting the source signal to a one dimensional tensor, resampling the source signal to a given frequency, normalizing the source signal between 0 and 1, standardizing the source signal, padding the ECG signal with zeros in case the ECG signal does not reach the expected length, replace NaN elements with zeros, remove an ECG signal wander line, remove a given frequency from the ECG signal, converting the one dimensional signal to a two dimensional scalogram (continuous wavelet transform), converting the one dimensional signal to a two dimensional spectrogram (Short time Fourier Transform), and/or converting the one dimensional signal to a mix of continuous wavelet transform and Short Time Fourier Transform (either summing up or concatenating). For example, the Short Time Fourier Transform may include analyzing the time and/or frequency domain for a portion of the one or more data subsets, and then performing a sliding of the one or more data subsets to reiterate the same analysis before recombining the resulting transforms. The continuous wavelet transform may include processing the entirety of the one or more data subsets at once, modulating the wavelet dilation and location, and performing a convolution between the resulting wavelet function and the one or more data subsets.

The method may also include determining, by the one or more processors and using a trained machine-learning model, one or more signal classifications for the one or more data subsets (Step 210). A machine-learning model, which may have been previously trained, may receive (e.g., may be “fed”) the one or more augmented data subsets of the canine. The machine-learning model may then analyze the one or more augmented data subsets. The machine-learning model may then output at least one signal classification for each of the one or more augmented data subsets. In some embodiments, the one or more signal classifications may include at least one of a normal classification, an abnormal classification, or a non-diagnostic classification. The normal classification may indicate that the analyzed data subset includes normal readings. For example, the data subset readings may be normal if the readings fall within a particular range. The abnormal classification may indicate that the analyzed data subset includes abnormal readings. For example, the data subset readings may be abnormal if the readings fall outside a particular range. The non-diagnostic classification may indicate that there is a possible error, where the data subset does not include normal readings or abnormal readings. In some embodiments, other signal classifications may include an unsuitable classification, a supraventricular classification, a ventricular classification, a bradyarrhythmia classification, and/or a conduction disorder classification, as described in the previous section.

In some embodiments, the trained machine-learning model may also process the canine metadata, in addition to the one or more data subsets, to determine one or more signal classifications. The machine-learning model may then analyze the one or more augmented data subsets and canine metadata. The machine-learning model may then output the at least one signal classification for each of the one or more augmented data subsets.

The method may also include aggregating, by the one or more processors, the one or more signal classifications to determine a result classification (Step 212). For example, the one or more signal classifications may be aggregated to determine the amount of normal and abnormal signal classifications. If the number of abnormal signal classifications exceeds a threshold, the result classification may include an abnormal classification result. If the number of abnormal signal classifications does not exceed a threshold, the result classification may include a normal result classification. In some embodiments, if the number of unsuitable classifications exceeds a threshold, the result classification may include an unsuitable result classification. Additionally, for example, if the number of supraventricular classifications exceeds a threshold, the result classification may include a supraventricular result classification. Additionally, for example, if the number of ventricular classifications exceeds a threshold, the result classification may include a ventricular result classification. Additionally, for example, if the number of bradyarrhythmia classifications exceeds a threshold, the result classification may include a bradyarrhythmia result classification. Additionally, for example, if the number of conduction disorder classifications exceeds a threshold, the result classification may include a conduction disorder classification.

In some embodiments, the aggregating may include using the canine metadata to determine whether the signal classification should be updated. For example, the one or more data subsets, one or more signal classifications, and canine metadata may be compared to stored canine ECG data. The stored canine ECG data may include stored ECG data subsets, signal classifications, and canine metadata. The method may include determining at least one stored ECG data subset that is associated with stored canine metadata that is similar to the canine metadata. For example, the stored canine metadata and the canine metadata may include the same gender and age range. The method may also include comparing the at least one stored ECG data subset with one of the data sequences to determine whether an associated signal classification should be updated. For example, a data subset may have an abnormal classification and may be associated with a female poodle that is 8 years old. The data subset may be compared to a stored data subset that corresponds to a female poodle that is 7 years old, but the stored data subset may have a normal classification. However, upon comparing the data subsets, it may be determined that the data subsets are identical and that the abnormal classification may be updated and changed to a normal classification.

The method may also include outputting, by the one or more processors, the result classification to an electronic storage device and/or a display (Step 214). In some embodiments, one or more user interfaces of the electronic storage device may display a normal result classification or an abnormal result classification. In some embodiments, one or more user interfaces of the electronic storage device may display an unsuitable result classification, a supraventricular result classification, a ventricular result classification, a bradyarrhythmia result classification, and/or a conduction disorder result classification. In some embodiments, the result classification may be output to the electronic storage device for storage and additional analysis. In some embodiments, a display of one or more user devices may display a normal result classification or an abnormal result classification. In some embodiments, a display of one or more user devices may display an unsuitable result classification, a supraventricular result classification, a ventricular result classification, a bradyarrhythmia result classification, and/or a conduction disorder result classification. In some embodiments, the outputting may include color-coding one or more of the of the data subsets according to the predicted signal classification. For example, if the predicted signal classification for a data subset is an abnormal classification, the data subset may be color-coded red. Additionally, if the predicted signal classification for a data subset is a normal classification, the data subset may be color-coded green. Additionally, if the predicted signal classification for a data subset is a non-diagnostic classification, the data subset may be color-coded blue.

The method may also include displaying, by the one or more processors, an alert indicating at least one of the one or more data subsets includes the abnormal classification. For example, if at least one of the one or more data subsets includes an abnormal classification, an alert may be displayed on one or more user interfaces, where the alert may indicate the data subset(s) that include an abnormal classification. In some embodiments, the alert may be displayed if the number of data subsets that include an abnormal classification exceeds a threshold. For example, if eight of the one or more data subsets include an abnormal classification, and the threshold is five, then an alert may be displayed indicating the eight data subsets that include the abnormal classification.

In some embodiments, the method may also include displaying, by the one or more processors, an alert indicating that at least one of the one or more data subsets includes an unsuitable classification, a supraventricular classification, a ventricular classification, a bradyarrhythmia classification, and/or a conduction disorder classification. For example, an alert may be displayed on one or more user interfaces if at least one of the one or more data subsets includes an unsuitable classification, a supraventricular classification, a ventricular classification, a bradyarrhythmia classification, and/or a conduction disorder classification, where the alert may indicate the data subset(s) that include the particular classification. In some embodiments, the alert may be displayed if the number of data subsets that include an unsuitable classification, a supraventricular classification, a ventricular classification, a bradyarrhythmia classification, and/or a conduction disorder classification exceeds a threshold. For example, if ten of the one or more data subsets include a supraventricular classification, and the threshold is nine, then an alert may be displayed indicating the ten data subsets that include the supraventricular classification.

Although FIG. 2 shows example blocks of exemplary method 200, in some implementations, the exemplary method 200 may include additional blocks, fewer blocks, different blocks, or differently arranged blocks than those depicted in FIG. 2. Additionally, or alternatively, two or more of the blocks of the exemplary method 200 may be performed in parallel.

### Exemplary Platform for Predicting an ECG Classification

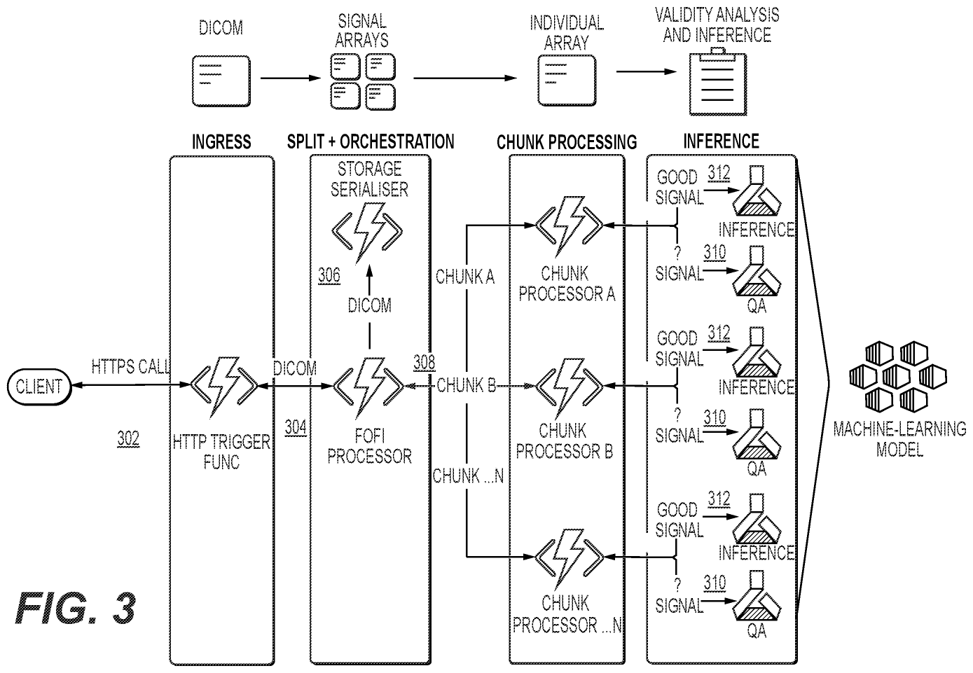

FIG. 3 depicts a process further illustrating a platform for predicting a classification of electrocardiogram (“ECG”) signals of a canine, according to one or more embodiments. Notably, process 300 may be performed by one or more processors of a server that is in communication with one or more user devices and other external system(s) via a network. However, it should be noted that process 300 may be performed by any one or more of the server, one or more user devices, or other external systems.

A platform may receive a DICOM file from a client, where the client may communicate the DICOM file utilizing one or more HTTPS calls (Step 302). For example, a HTTP trigger function may invoke a function with a HTTP request, where the function may include receiving and/or requesting the DICOM file. The DICOM file may include canine ECG data that includes one or more ECG signals. The client may include any web-capable process.

The platform may then analyze and transform the DICOM file (Step 304). For example, the platform may analyze and transform at least one ECG signal included in the DICOM file. Additionally, analyzing the DICOM file may include storing the DICOM file in a storage serialiser for future use (Step 306).

The platform may extract at least one ECG signal from the DICOM file, and then segment the at least one ECG signal into one or more data chunks and process the one or more data chunks (Step 308). The platform may utilize a fan-out-fan-in (FOFI) processor to analyze the DICOM file. Additionally, the platform may utilize one or more processors to extract the at least one ECG signal from the DICOM file. The platform may segment the at least one ECG signal based on a variety of factors of the ECG data, such as a signal length, a signal type, and/or a signal interval. Also at Step 308, one or more transformations may be applied to the ECG signal to improve the signal quality.

The platform may then perform a validity analysis (e.g., quality analysis) on each of the one or more data chunks (Step 310). For example, the one or more processors may perform a validity analysis (e.g., quality analysis) on the one or more data chunks to determine whether each of the one or more data chunks has a good signal. Additionally, the one or more processors may remove one or more data chunks that does not have a good signal.

The one or more data chunks that pass the validity analysis may then be sent to one or more machine-learning models for processing to determine a classification for each of the one or more data chunks (Step 312). The platform may send the one or more data chunks that pass the validity analysis to one or more machine-learning models. Upon processing the one or more data chunks, the one or more machine-learning models may output at least one classification for each of the one or more data chunks. Example classifications may include a normal classification, an abnormal classification, and/or a non-diagnostic classification. By way of further example, additional classifications may include an unsuitable classification, a supraventricular classification, a ventricular classification, a bradyarrhythmia classification, and/or a conduction disorder classification.

### Exemplary Environment

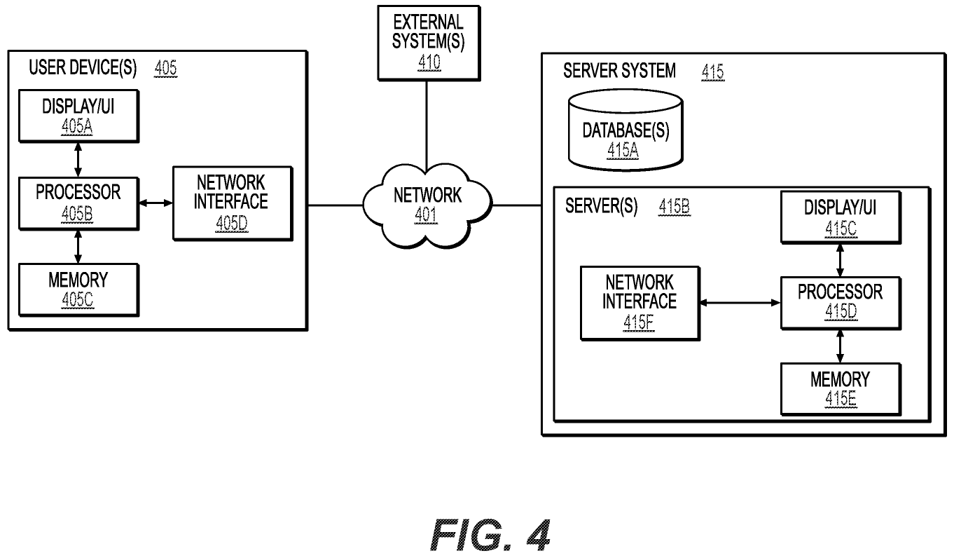

FIG. 4 depicts an exemplary environment 400 that may be utilized with techniques presented herein. One or more user device(s) 405, one or more external system(s) 410, and one or more server system(s) 415 may communicate across a network 401. As will be discussed in further detail below, one or more server system(s) 415 may communicate with one or more of the other components of the environment 400 across network 401. The one or more user device(s) 405 may be associated with a user, e.g., a user associated with one or more of generating, training, or tuning a machine-learning model for predicting a classification of canine ECG data.

In some embodiments, the components of the environment 400 are associated with a common entity, e.g., a veterinarian, clinic, animal specialist, research center, or the like. In some embodiments, one or more of the components of the environment is associated with a different entity than another. The systems and devices of the environment 400 may communicate in any arrangement. As will be discussed herein, systems and/or devices of the environment 400 may communicate in order to one or more of generate, train, and/or use a machine-learning model to predict a classification of canine ECG data, among other activities.

The user device 405 may be configured to enable the user to access and/or interact with other systems in the environment 400. For example, the user device 405 may be a computer system such as, for example, a desktop computer, a mobile device, a tablet, etc. In some embodiments, the user device 405 may include one or more electronic application(s), e.g., a program, plugin, browser extension, etc., installed on a memory of the user device 405.

The user device 405 may include a display/user interface (UI) 405A, a processor 405B, a memory 405C, and/or a network interface 405D. The user device 405 may execute, by the processor 405B, an operating system (O/S) and at least one electronic application (each stored in memory 405C). The electronic application may be a desktop program, a browser program, a web client, or a mobile application program (which may also be a browser program in a mobile O/S), an applicant specific program, system control software, system monitoring software, software development tools, or the like. For example, environment 400 may extend information on a web client that may be accessed through a web browser. In some embodiments, the electronic application(s) may be associated with one or more of the other components in the environment 400. The application may manage the memory 405C, such as a database, to transmit streaming data to network 401. The display/UI 405A may be a touch screen or a display with other input systems (e.g., mouse, keyboard, etc.) so that the user(s) may interact with the application and/or the O/S. The network interface 405D may be a TCP/IP network interface for, e.g., Ethernet or wireless communications with the network 401. The processor 405B, while executing the application, may generate data and/or receive user inputs from the display/UI 405A and/or receive/transmit messages to the server system 415, and may further perform one or more operations prior to providing an output to the network 401.

External systems 410 may be, for example, one or more third party and/or auxiliary systems that integrate and/or communicate with the server system 415 in performing various document information extraction tasks. External systems 410 may be in communication with other device(s) or system(s) in the environment 400 over the one or more networks 401. For example, external systems 410 may communicate with the server system 415 via API (application programming interface) access over the one or more networks 401, and also communicate with the user device(s) 405 via web browser access over the one or more networks 401.

In various embodiments, the network 401 may be a wide area network (“WAN”), a local area network (“LAN”), a personal area network (“PAN”), or the like. In some embodiments, network 401 includes the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system 415 may include an electronic data system, e.g., a computer-readable memory such as a hard drive, flash drive, disk, etc. In some embodiments, the server system 415 includes and/or interacts with an application programming interface for exchanging data to other systems, e.g., one or more of the other components of the environment.

The server system 415 may include a database 415A and at least one server 415B. The server system 415 may be a computer, system of computers (e.g., rack server(s)), and/or or a cloud service computer system. The server system may store or have access to database 415A (e.g., hosted on a third party server or in memory 415E). The server(s) may include a display/UI 415C, a processor 415D, a memory 415E, and/or a network interface 415F. The display/UI 415C may be a touch screen or a display with other input systems (e.g., mouse, keyboard, etc.) for an operator of the server 415B to control the functions of the server 415B. The server system 415 may execute, by the processor 415D, an operating system (O/S) and at least one instance of a servlet program (each stored in memory 415E).

The server system 415 may generate, store, train, or use a machine-learning model, configured to predict a classification of canine ECG data. The server system 415 may include a machine-learning model and/or instructions associated with the machine-learning model, e.g., instructions for generating a machine-learning model, training the machine-learning model, using the machine-learning model, etc. The server system 415 may include training data, e.g., canine ECG data, at least one signal classification, and/or canine metadata.

In some embodiments, a system or device other than the server system 415 is used to generate and/or train the machine-learning model. For example, such a system may include instructions for generating the machine-learning model, the training data and ground truth, and/or instructions for training the machine-learning model. A resulting trained machine-learning model may then be provided to the server system 415.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn associations between the canine ECG data, the signal classifications, and/or the canine metadata, such that the trained machine-learning model is configured to determine a signal classification in response to the input canine ECG data based on the learned associations.

In various embodiments, the variables of a machine-learning model may be interrelated in any suitable arrangement in order to generate the output. For example, in some embodiments, the machine-learning model may include signal processing architecture that is configured to identify, isolate, and/or extract features, patterns, and/or structure in a text. For example, the machine-learning model may include one or more convolutional neural network (“CNN”) configured to identify features in the document information data, and may include further architecture, e.g., a connected layer, neural network, etc., configured to determine one or more classifications of canine ECG data.

Although depicted as separate components in FIG. 4, it should be understood that a component or portion of a component in the environment 400 may, in some embodiments, be integrated with or incorporated into one or more other components. For example, a portion of the display 415C may be integrated into the user device 405 or the like. In some embodiments, operations or aspects of one or more of the components discussed above may be distributed amongst one or more other components. Any suitable arrangement and/or integration of the various systems and devices of the environment 400 may be used.

Further aspects of the machine-learning model and/or how it may be utilized to predict at least one classification of ECG data are discussed in further detail in the methods above. In these methods, various acts may be described as performed or executed by a component from FIG. 4, such as the server system 415, the user device 405, or components thereof. However, it should be understood that in various embodiments, various components of the environment 400 discussed above may execute instructions or perform acts including the acts discussed above and below. An act performed by a device may be considered to be performed by a processor, actuator, or the like associated with that device. Further, it should be understood that in various embodiments, various steps may be added, omitted, and/or rearranged in any suitable manner.

In general, any process or operation discussed in this disclosure that is understood to be computer-implementable, such as the processes illustrated in FIGS. 1-3, may be performed by one or more processors of a computer system, such any of the systems or devices in the environment 400 of FIG. 4, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 4. One or more processors of a computer system may be included in a single computing device or distributed among a plurality of computing devices. A memory of the computer system may include the respective memory of each computing device of the plurality of computing devices.

### Exemplary Device

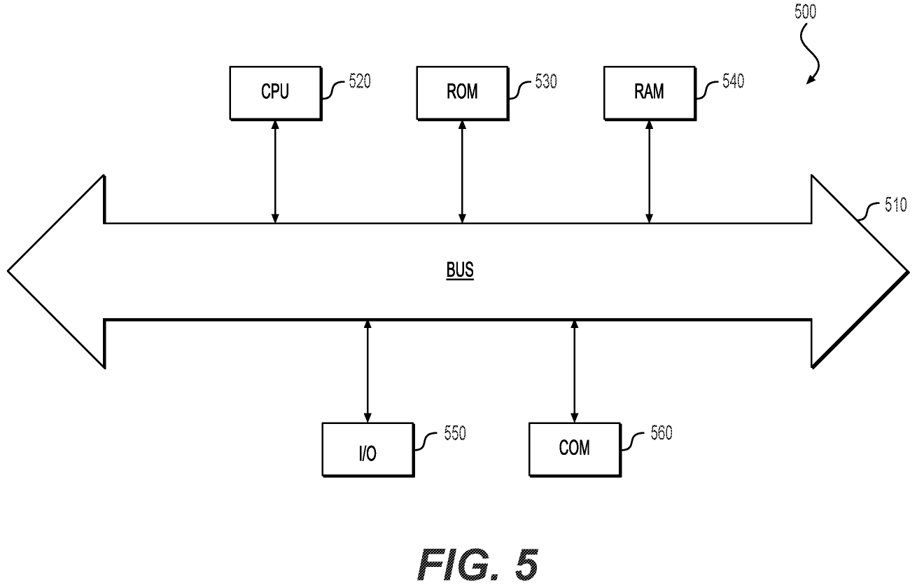

FIG. 5 is a simplified functional block diagram of a computer 500 that may be configured as a device for executing the methods and processes of FIGS. 1-3, according to exemplary embodiments of the present disclosure. For example, device 500 may include a central processing unit (CPU) 520. CPU 520 may be any type of processor device including, for example, any type of special purpose or a general-purpose microprocessor device. As will be appreciated by persons skilled in the relevant art, CPU 520 also may be a single processor in a multi-core/multiprocessor system, such system operating alone, or in a cluster of computing devices operating in a cluster or server farm. CPU 520 may be connected to a data communication infrastructure 510, for example, a bus, message queue, network, or multi-core message-passing scheme.

Device 500 also may include a main memory 540, for example, random access memory (RAM), and also may include a secondary memory 530. Secondary memory 530, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 530 may include other similar means for allowing computer programs or other instructions to be loaded into device 500. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 500.

Device 500 also may include a communications interface (“COM”) 560. Communications interface 560 allows software and data to be transferred between device 500 and external devices. Communications interface 560 may include a modem, a network interface (such as an Ethernet card), a communications port, a PCMCIA slot and card, or the like. Software and data transferred via communications interface 560 may be in the form of signals, which may be electronic, electromagnetic, optical, or other signals capable of being received by communications interface 560. These signals may be provided to communications interface 560 via a communications path of device 500, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 500 also may include input and output ports 550 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the general description and the detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

As used herein, a term such as “user” or the like generally encompasses a pet parent and/or pet parents. A term such as “canine” or “pet” or the like generally encompasses a user's pet, such as a dog, where the term may encompass multiple pets.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model/system is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as linear regression, logistical regression, random forest, gradient boosted machine (GBM), decision tree, gradient boosting in a decision tree, deep learning, and/or a deep neural network. Supervised and/or unsupervised training may be employed. For example, supervised learning may include providing training data and classifications corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for classifying electrocardiogram signals of a canine, the method comprising:
receiving, by one or more processors, electrocardiogram data of a canine, the electrocardiogram data including at least one electrocardiogram signal;
segmenting, by the one or more processors, the electrocardiogram data into one or more data subsets;
pre-processing, by the one or more processors, the one or more data subsets, the pre-processing including excluding the one or more data subsets that include a poor electrocardiogram signal;
augmenting, by the one or more processors, the one or more data subsets;
determining, by the one or more processors and using a trained machine-learning model, one or more signal classifications for the one or more data subsets;
aggregating, by the one or more processors, the one or more signal classifications to determine a result classification; and
outputting, by the one or more processors, the result classification to an electronic storage device and/or a display.

2. The computer-implemented method of claim 1, the method further comprising:
receiving, by the one or more processors, canine metadata associated with the electrocardiogram data; and
analyzing, by the one or more processors and using the trained machine-learning model, the one or more signal classifications and the canine metadata to determine whether to update the one or more signal classifications.

3. The computer-implemented method of claim 2, wherein the canine metadata includes at least one of a breed, an age, a gender, and a weight.

4. The computer-implemented method of claim 1, wherein the pre-processing the one or more data subsets includes at least one of a baseline wander removal, a signal normalization, a frequency removal, a heart rate computation, or an unsuitable signal removal.

5. The computer-implemented method of claim 1, the augmenting including:
applying, by the one or more processors, at least one transformation to the one or more data subsets.

6. The computer-implemented method of claim 1, wherein the receiving includes receiving at least one Digital Imaging and Communications in Medicine (DICOM) file that includes the electrocardiogram data.

7. The computer-implemented method of claim 1, the method further comprising:
in response to receiving the electrocardiogram data, storing, by the one or more processors, the electrocardiogram data in the electronic storage device.

8. The computer-implemented method of claim 1, wherein the one or more data subsets include an electrocardiogram signal length of eight seconds.

9. The computer-implemented method of claim 1, wherein the one or more signal classifications include at least one of a normal classification or an abnormal classification.

10. The computer-implemented method of claim 9, the method further comprising:
displaying, by the one or more processors, an alert indicating at least one of the one or more data subsets includes the abnormal classification.

11. A computer system for classifying electrocardiogram signals of a canine, the computer system comprising:
at least one memory storing instructions; and
at least one processor configured to execute the instructions to perform operations comprising:
receiving electrocardiogram data of a canine, the electrocardiogram data including at least one electrocardiogram signal;
segmenting the electrocardiogram data into one or more data subsets;
pre-processing the one or more data subsets, the pre-processing including excluding the one or more data subsets that include a poor electrocardiogram signal;
augmenting the one or more data subsets;
determining, using a trained machine-learning model, one or more signal classifications for the one or more data subsets;
aggregating the one or more signal classifications to determine a result classification; and
outputting the result classification to an electronic storage device and/or a display.

12. The computer system of claim 11, the operations further comprising:
receiving canine metadata associated with the electrocardiogram data; and
analyzing, by the one or more processors and using the trained machine-learning model, the one or more signal classifications and the canine metadata to determine whether to update the one or more signal classifications.

13. The computer system of claim 12, wherein the canine metadata includes at least one of a breed, an age, a gender, and a weight.

14. The computer system of claim 11, wherein the pre-processing the one or more data subsets includes at least one of a baseline wander removal, a signal normalization, a frequency removal, a heart rate computation, or an unsuitable signal removal.

15. The computer system of claim 11, the augmenting including:
applying at least one transformation to the one or more data subsets.

16. The computer system of claim 11, wherein the receiving includes receiving at least one Digital Imaging and Communications in Medicine (DICOM) file that includes the electrocardiogram data.

17. The computer system of claim 11, wherein the one or more data subsets include an electrocardiogram signal length of eight seconds.

18. A non-transitory computer-readable medium storing instructions that, when executed by a processor, cause the processor to perform operations for classifying electrocardiogram signals of a canine, the operations comprising:
receiving electrocardiogram data of a canine, the electrocardiogram data including at least one electrocardiogram signal;
segmenting the electrocardiogram data into one or more data subsets;
pre-processing the one or more data subsets, the pre-processing including excluding the one or more data subsets that have a poor electrocardiogram signal;
augmenting the one or more data subsets;
determining, using a trained machine-learning model, one or more signal classifications for the one or more data subsets;
aggregating the one or more signal classifications to determine a result classification; and
outputting the result classification to an electronic storage device and/or a display.

19. The non-transitory computer-readable medium of claim 18, wherein the one or more data subsets include an electrocardiogram signal length of eight seconds.

20. The non-transitory computer-readable medium of claim 18, wherein the one or more signal classifications include at least one of a normal classification, an abnormal classification, or a non-diagnostic classification.
